(19) United States
(12) Patent Application Publication
(10) Pub. No.: US 2025/0259439 A1
(43) Pub. Date: Aug. 14, 2025

(54) INSPECTION ASSISTANCE SYSTEM, INSPECTION ASSISTANCE METHOD, CONTROL METHOD FOR MEDICAL DEVICE, AND COMPUTER-READABLE RECORDING MEDIUM ON WHICH PROGRAM IS RECORDED

(71) Applicant: OLYMPUS MEDICAL SYSTEMS CORP., Tokyo (JP)

(72) Inventors: Naoki FUKATSU, Tokyo (JP); Kensuke MIYAKE, Tokyo (JP); Hiromasa FUJITA, Tokyo (JP)

(73) Assignee: OLYMPUS MEDICAL SYSTEMS CORP., Tokyo (JP)

(21) Appl. No.: 19/169,460

(22) Filed: Apr. 3, 2025

Related U.S. Application Data
(63) Continuation of application No. PCT/JP2022/037163, filed on Oct. 4, 2022.

(51) Int. Cl. G06V 10/98; A61B 1/00; G06V 10/70; G06V 20/40; G16H 40/67

(52) U.S. Cl. CPC G06V 10/993; A61B 1/000096; A61B 1/00055; A61B 1/00059; G06V 10/70; G06V 20/40; G16H 40/67; G06V 2201/03

(57) ABSTRACT

An inspection assistance system includes one or more processors including hardware, and the one or more processors are configured to: acquire a video signal including information of a medical image; perform recognition processing based on the video signal, to enable outputting of inspection assistance information for assisting an inspection; acquire identification information included in the video signal, the identification information being different from the information of the medical image; and determine whether the video signal is a video signal for the recognition processing, based on the identification information.

### CROSS REFERENCE TO RELATED APPLICATION

This application is a continuation application of PCT/JP2022/037163 filed on Oct. 4, 2022, the entire contents of which are incorporated herein by this reference.

### BACKGROUND OF THE INVENTION

### 1. Field of the Invention

The present disclosure relates to an inspection assistance system, an inspection assistance method, a control method for a medical device, and a computer-readable recording medium on which a program is recorded, which are preferable for a device configured to perform an AI determination.

### 2. Description of the Related Art

In recent years, medical systems that use an AI (artificial intelligence) for performing various determinations have been developed also in medical fields. For example, in a colonoscopy, an endoscope AI operation assistance system is sometimes employed to perform a high-quality endoscopy with a reduced burden on a patient. The system is configured to judge a current endoscopy status by using an AI, and present assistance information fitted to the status.

For enabling a highly-accurate AI determination, an endoscopic image suitable for the AI determination is required to be used. However, even if an endoscopic image suitable for the AI determination is picked up, if an erroneous wiring or the like occurs between a video processor apparatus of an endoscope system and an apparatus configured to output operation assistance information and the like by an AI, based on the endoscopic image (hereinafter, referred to as an inspection assistance apparatus), the AI determination is not performed based on the suitable endoscopic image.

As a technology for preventing such an erroneous wiring, Japanese Patent Application Laid-Open Publication No. 2010-268063 discloses a technology for authenticating a device as a connection party in order to manage whether to permit information communication between electronic devices. In the technology, when an image forming apparatus detects a connection of an external apparatus, the image forming apparatus acquires an external apparatus ID of the external apparatus, and permits the use of the external apparatus if the acquired external apparatus ID matches an external apparatus ID stored in advance.

This technology is applied to a video processor apparatus and an inspection assistance apparatus, to enable confirmation whether a connection destination device of the communication connection is correct.

Note that, between the video processor apparatus and the inspection assistance apparatus, two types of connections are used. The two types of connections include a communication connection for transmitting identification signals and control signals, and a video connection for transmitting video signals of endoscopic images. In addition, there is a case where the video processor apparatus is configured to be capable of outputting a video output from a plurality of types of output ports for respective image transmission formats.

### SUMMARY OF THE INVENTION

An inspection assistance system according to one aspect of the present disclosure includes one or more processors including hardware. The one or more processors are configured to: acquire a video signal including information of a medical image; perform recognition processing based on the video signal, to enable outputting of inspection assistance information for assisting an inspection; acquire identification information included in the video signal, the identification information being different from the information of the medical image; and determine whether the video signal is a video signal for the recognition processing, based on the identification information.

An inspection assistance method according to one aspect of the present disclosure includes: acquiring a video signal including information of a medical image; acquiring identification information included in the video signal, the identification information being different from the information of the medical image; determining whether the video signal is a video signal for recognition processing based on the identification information; and performing the recognition processing based on the video signal, to output inspection assistance information for assisting an inspection using the medical image.

A control method for a medical device according to one aspect of the present disclosure includes: acquiring transmission source device information related to a transmission source device which is a transmission source of a medical image: determining whether the transmission source device as a connection destination is suitable, based on the transmission source device information; acquiring a video signal including information of the medical image; performing recognition processing based on the video signal, to enable outputting of inspection assistance information for assisting an inspection; and determining, based on the video signal, whether the video signal is a video signal for the recognition processing.

A computer-readable recording medium on which a program is recorded according to one aspect of the present disclosure records the program causing a computer to execute procedures for: acquiring transmission source device information related to a transmission source device which is a transmission source of a medical image; determining whether the transmission source device as a connection destination is suitable, based on the transmission source device information; acquiring a video signal including information of the medical image; performing recognition processing based on the video signal, to enable outputting of inspection assistance information for assisting an inspection; and determining, based on the video signal, whether the video signal is a video signal for the recognition processing.

### BRIEF DESCRIPTION OF THE DRAWINGS

FIG. 1 is a configuration diagram showing an inspection assistance system according to a first embodiment of the present disclosure.

FIG. 2 is an explanatory diagram for describing one example of a determination method for an input eligibility determination by an AI information confirmation section 13b.

FIG. 3 is an explanatory diagram for describing another example of the determination method for the input eligibility determination by the AI information confirmation section 13b.

FIG. 4 is a flowchart showing an AI determination control by the AI information confirmation section 13b.

FIG. 5 is a flowchart showing a device correctness determination by a connection destination confirmation section 13a.

FIG. 6 is a flowchart showing the device correctness determination by the connection destination confirmation section 13a.

FIG. 7 is a block diagram showing a modified example.

### DETAILED DESCRIPTION OF THE PREFERRED EMBODIMENT(S)

Hereinafter an embodiment of the present disclosure will be described in detail with reference to drawings.

### First Embodiment

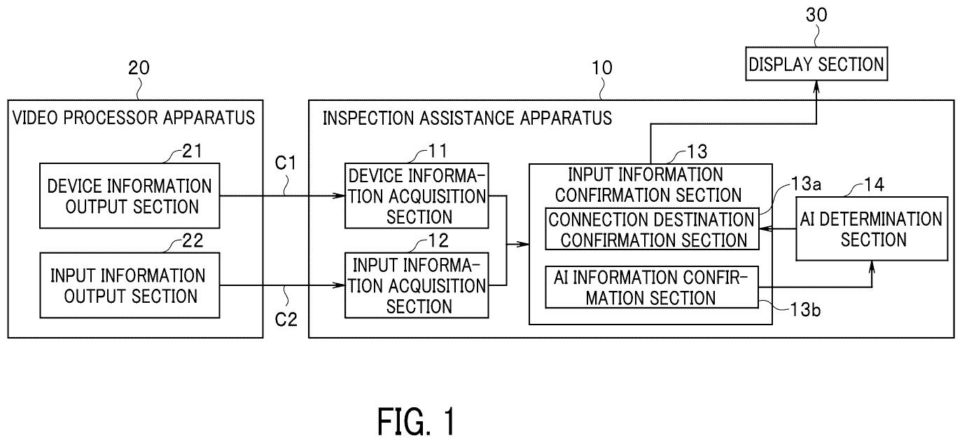

FIG. 1 is a configuration diagram showing an inspection assistance system according to a first embodiment of the present disclosure. The present embodiment enables checking that a medical image suitable for an AI determination is inputted into an inspection assistance apparatus.

In the present embodiment, the inspection assistance system is applied to an endoscopy, but not limited to the endoscopy. The inspection assistance system is applicable to various kinds of AI processing that adopt AI determinations. For example, the inspection assistance system can be applied to AI processing for outputting endoscope operation guide information based on endoscopic images, AI processing for outputting control information of an automatic insertion endoscope based on endoscopic images, and AI processing for outputting identification information of a lesion and a site of an organ based on endoscopic images.

FIG. 1 shows configurations of an inspection assistance apparatus 10 and a video processor apparatus 20 that constitute the inspection assistance system according to the first embodiment of the present disclosure. The video processor apparatus 20 is configured to output information to be used for an AI determination to the inspection assistance apparatus 10.

The video processor apparatus 20 includes a device information output section 21 and an input information output section 22. To the video processor apparatus 20, for example, an endoscope, not shown, which is configured to be inserted into a subject and pick up endoscopic images inside a body cavity, may be connected, and the endoscopic images such as endoscopic inspection images may be supplied from the endoscope to the video processor apparatus 20. The video processor apparatus 20 is capable of acquiring not only the endoscopic images but also various kinds of information. For example, the video processor apparatus 20 may be configured to be capable of acquiring information on whether the endoscope has been connected to the video processor apparatus 20 and information on whether an inspection by the endoscope has been started. Furthermore, the video processor apparatus 20 may be configured to acquire information related to switching of observation modes of the endoscope, for example, various observation modes such as an NBI (Narrow Band Imaging) mode, an RDI (Red Dichromatic Imaging) mode.

The device information output section 21 is configured to transmit transmission source device information via a first communication cable C1 configuring a first communication route. The transmission source device information is information related to a device which is a transmission source (transmission source device) of input information to be inputted into the inspection assistance apparatus 10, that is, information related to the video processor apparatus 20 which is a connection destination of the inspection assistance apparatus 10. Note that the transmission source device information transmitted by the device information output section 21 may include, for example, not only ID information for identifying the video processor apparatus 20 but also information related to an action of the video processor apparatus 20 (action information), information related to an operation of the video processor apparatus 20 (operation information), information related to a video format of a video signal outputted from the video processor apparatus 20, etc. The action information and the operation information in the transmission source device information include the information on whether the endoscope has been connected to the video processor apparatus 20, the information on whether the inspection by the endoscope has been started, the information related to the switching of the observation modes of the endoscope, etc.

The input information output section 22 outputs the input information to be inputted to the inspection assistance apparatus 10 via a second communication cable C2 configuring a second communication route. In other words, the input information output section 22 outputs a video signal of an image for an AI determination, for example, an image including an endoscopic image such as an endoscopic inspection image, as the input information to be inputted into the inspection assistance apparatus 10. Note that the image based on the video signal outputted by the input information output section 22 includes, in a part of regions in the image, an endoscopic image as a medical image.

Note that description has been made that the first communication route and the second communication route are configured respectively with the first communication cable C1 and the second communication cable C2, but not limited thereto. The first and second communication routes may be configured with wireless routes.

The inspection assistance apparatus 10 includes a device information acquisition section 11, an input information acquisition section 12, an input information confirmation section 13, and an AI determination section 14. The entirety of the inspection assistance apparatus 10 or at least one of the device information acquisition section 11, the input information acquisition section 12, the input information confirmation section 13, and the AI determination section 14 may be configured by one or more processors using a CPU (Central Processing Unit), an FPGA (Field Programmable Gate Array) or the like, and may operate according to a program stored in a memory, not shown, to control respective sections, or may realize a part of or all of the functions of the respective sections by an electronic circuit of hardware.

The device information acquisition section 11 acquires the transmission source device information transmitted from the device information output section 21 via the first communication route, and outputs the acquired transmission source device information to the input information confirmation section 13. The input information acquisition section 12 acquires the input information transmitted from the input information output section 22 via the second communication route, and outputs the acquired input information to the input information confirmation section 13.

The input information confirmation section 13 includes a connection destination confirmation section 13a and an AI information confirmation section 13b. The connection destination confirmation section 13a receives the transmission source device information from the device information output section 21. The connection destination confirmation section 13a uses the transmission source device information, to perform a device correctness determination and confirm the correctness of the device. The device correctness determination is a determination on whether the device which is the connection destination, that is, the device of the transmission source of the endoscopic image which is the input information for determining the contents of assistance in the inspection assistance apparatus 10 is a correct device.

The AI information confirmation section 13b receives the input information from the input information output section 22. The AI information confirmation section 13b performs an input eligibility determination on whether the input information is suitable, for example, for predetermined recognition processing such as the AI determination to output an input eligibility determination result, which is a determination result, to the AI determination section 14.

The AI determination section 14 receives the input information, and the input eligibility determination result from the AI information confirmation section 13b. The AI determination section 14 decides whether to use the input information for the AI determination, based on the input eligibility determination result. Thus, if the AI determination section 14 receives, from the AI information confirmation section 13b, the input eligibility determination result that the input information is suitable for the recognition processing, the AI determination section 14 performs the recognition processing such as the AI determination based on the input information. The AI determination section 14 outputs the determination result of the AI determination, which is the recognition result of the recognition processing, to the connection destination confirmation section 13a. In addition, if the AI determination section 14 receives, from the AI information confirmation section 13b, the input eligibility determination result that the input information is not suitable for the recognition processing, the AI determination section 14 does not perform the recognition processing such as the AI determination based on the input information.

Note that the AI information confirmation section 13b may supply the input information to the AI determination section 14 only in the case where the input eligibility determination result that the input information is suitable for the recognition processing is obtained.

When receiving the AI determination result from the AI determination section 14, the connection destination confirmation section 13a decides whether to adopt the AI determination result based on the determination result of the device correctness for the device as the transmission source of the input information used for the AI determination. In other words, when having confirmed that the device is correct based on the determination result of the device correctness, the connection destination confirmation section 13a adopts the AI determination result and outputs the AI determination result to a display section 30. Furthermore, when having confirmed that the device is not correct based on the determination result of the device correctness, the connection destination confirmation section 13a does not output the AI determination result to the display section 30.

The display section 30 displays, on a display screen thereof, the information from the input information confirmation section 13. When receiving the input of the AI determination result, the display section 30 displays the inputted AI determination result on the display screen. The input information confirmation section 13 may provide the determination result of the device correctness and the determination result of the input eligibility to the display section 30, to display the results on the display section 30.

### Input Eligibility Determination

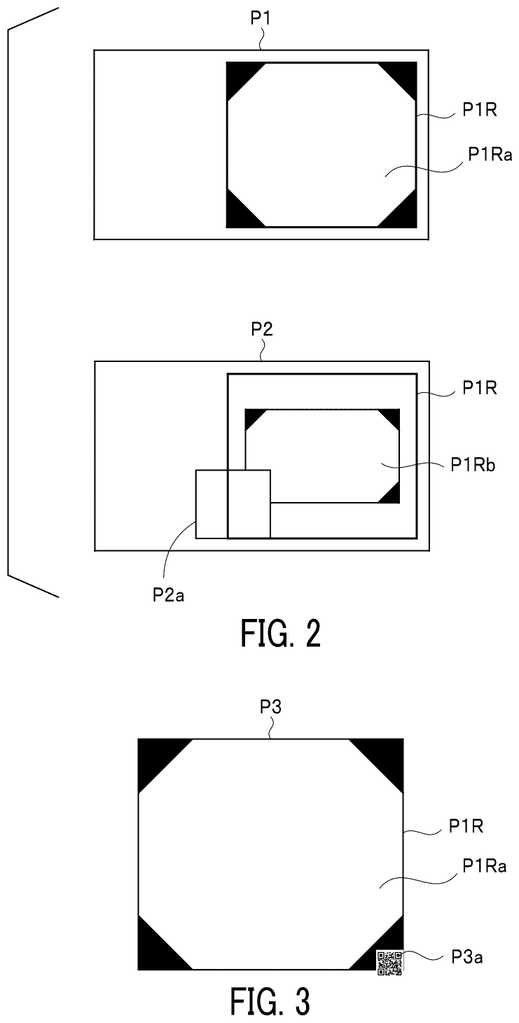

FIG. 2 and FIG. 3 are explanatory diagrams each for describing an example of a determination method for the input eligibility determination by the AI information confirmation section 13b.

The video processor apparatus 20 performs predetermined video signal processing on an image pickup signal obtained by photographing with the endoscope, not shown, to generate a video signal in a predetermined video format in which video parameters such as a resolution, a color gamut, a frame rate are set as appropriate. The video processor apparatus 20 encodes the generated video signal using a predetermined codec such as MPEG-4, H.265/HEVC, then containerizes the encoded video signal in various file formats such as AVI, MOV, WMV, FLV, or other file formats, and transmits the containerized video signal, via an output terminal that conforms to a predetermined transmission standard such as DVI or SDI. Note that the video format of the video signal that can be transmitted is defined according to the transmission standard.

If the video format that is not suitable for the AI determination is adopted, a highly-reliable AI determination result cannot be obtained. In addition, even if an image in the video format suitable for the AI determination is inputted into the video processor apparatus 20, there is a case where an image which is not suitable for the AI determination is transmitted to the inspection assistance apparatus 10 due to an erroneous wiring, or other factors. Also in such a case, a highly-reliable AI determination result cannot be obtained.

An image P1 in FIG. 2 shows one example of an image suitable for the AI determination by the AI determination section 14, and the image P1 is obtained by converting the video signal outputted from the input information output section 22 into an image. The image P1 includes, in a relatively large range of an entire region of the image, a display area P1R for displaying an endoscopic image. The display area P1R includes, inside thereof, an endoscopic image P1Ra, an effective image region of which has an octagonal shape by triangular masked regions (filled regions) being formed at four corners of the endoscopic image P1Ra. The endoscopic image P1Ra has a relatively high resolution. The AI determination section 14 performs recognition processing on the endoscopic image P1Ra, to obtain the AI determination result.

On the other hand, an image P2 in FIG. 2 shows one example of an image which is not suitable for the AI determination by the AI determination section 14. For example, in a case where a type of the endoscope connected to the video processor apparatus 20 is not appropriate, or in a case where the setting of the video processor apparatus 20 is not correct, if the video signal from the input information output section 22 is converted into an image, it is supposed that the image P2 will be obtained. In other words, the image P2 includes, in a display area P1R occupying a relatively large range of the entire region of the image, an endoscopic image P1Rb, an effective image region of which has an octagonal shape by triangular masked regions (filled regions) being formed at four corners of the endoscopic image P1Rb. The endoscopic image P1Rb has an aspect ratio different from that of the endoscopic image P1Ra. In other words, the endoscopic image P1Ra and the endoscopic image P1Rb are different from each other in the positions in the image based on the video signal as the input information, and the AI determination section 14 may not necessarily be able to obtain a correct AI determination result, even if performing the recognition processing on the endoscopic image P1Rb.

In addition, in a case where a wrong endoscope is connected to the video processor apparatus 20, in a case where a wrong setting is performed for the video processor apparatus 20, or in a case where an image which is not for the AI determination, for example, an image for printing, is inputted into the inspection assistance apparatus 10, or the like, the resolution of the endoscopic image P1Rb inputted into the inspection assistance apparatus 10 is sometimes lower than the resolution of the endoscopic image P1Ra. In this case, the AI determination section 14 may not necessarily be able to obtain a correct AI determination result, even if performing the recognition processing on the endoscopic image P1Rb. Furthermore, in a case where an image to be outputted to another apparatus, not shown, is erroneously provided to the inspection assistance apparatus 10, or the like, another image P2a may possibly be superimposed as a PIP (picture-in-picture) on a part of the region of the endoscopic image P1Rb. Also in this case, the AI determination section 14 may not be able to obtain a correct AI determination result.

The AI information confirmation section 13b may determine that the video signal is not the video signal for the recognition processing in at least one of: a case where at least one of an aspect ratio or an image quality of the medical image as the input information is different from an aspect ratio or an image quality of a medical image for learning used for generating an inference model of the AI determination section 14; or a case where the medical image includes another image superimposed thereon.

For such a determination, the AI information confirmation section 13b may include, for example, a memory, not shown, that stores information related to the features of the image suitable for the AI determination. The AI information confirmation section 13b performs an image analysis of the input information and an analysis of metadata accompanying the input information, to compare the information stored in the memory and the input information with regard to the features including an image quality format, a frame rate, the aspect ratio, superimposition of other information, etc., to determine the eligibility of the input information, and thereby obtains the input eligibility determination result. For example, the AI information confirmation section 13b may determine the position of the medical image in the image obtained based on the video signal as the input information, and may obtain the input eligibility determination result according to a degree of match between the determined position and the position of the medical image in the image suitable for the AI determination.

FIG. 2 describes the example in which the eligibility of the input information is determined by the image analysis of the endoscopic image which is the input information, or other analysis. On the other hand, FIG. 3 shows an example in which information directly indicating the eligibility of the input information is included in the input information.

An image P3 in FIG. 3 includes, in a display area P1R, identification information P3a in a superimposed manner. The identification information P3a indicates that the endoscopic image P1Ra is an image suitable for the AI determination. Note that, in the example shown in FIG. 3, the identification information P3a is image information. The identification information P3a is arranged in one of masked regions located at four corners of the display area P1R. Therefore, the information of the effective image region of the endoscopic image P1Ra is hardly lost by the identification information P3a.

As the identification information P3a, for example, a barcode or a QR code (registered trademark) may be used. Note that FIG. 3 shows the example in which the identification information P3a can be visually recognizable, but the one which is not visually recognizable may also be used as the identification information P3a.

In addition, the identification information P3a may not only be included, as an image, in the image based on the video signal, but also be included in header information of the video signal, or may be metadata, which indicates that the endoscopic image P1Ra is the image suitable for the AI determination, included in the input information.

The AI information confirmation section 13b can determine the eligibility of the input information by confirming the identification information P3a. Thus, the AI information confirmation section 13b determines, based on the video signal, whether the video signal is suitable for the recognition processing such as the AI determination.

Note that the AI information confirmation section 13b performs the input eligibility determination at an appropriate timing. For example, the AI information confirmation section 13b may perform the input eligibility determination at the time of activating the inspection assistance apparatus 10, or at the time when the endoscope, not shown, is connected to the video processor apparatus 20. In addition, the AI information confirmation section 13b may perform the input eligibility determination at the time of changing the setting of the video processor apparatus 20 that supplies the input information to the inspection assistance apparatus 10, or at a predetermined cycle.

When the AI information confirmation section 13b determines that the input information is not suitable for the AI determination based on the input eligibility determination result, the AI information confirmation section 13b may generate display information to supply the generated display information to the display section 30. The display information is for causing warning messages (alert) to be displayed on the display screen of the display section 30, and examples of the messages include “This is not the target endoscope”, “The setting is different”, and the like.

### Device Correctness Determination

The connection destination confirmation section 13a may include a memory that stores ID information related to a correct device, for determination of the device correctness. The connection destination confirmation section 13a may determine the device correctness by comparing the information stored in the memory and the transmission source device information. For example, when endoscopic images are acquired respectively in a plurality of inspection rooms, it can be considered that the endoscopic image, which has been acquired in the inspection room different from the target inspection room, is inputted, as the input information, from the video processor apparatus 20 to the AI information confirmation section 13b, due to the erroneous wiring, or other factors. Also in such a case, the AI determination for the input image is obtained correctly. Therefore, if the transmission source device information is not confirmed, the operation assistance is performed based on the endoscopic image obtained in the inspection room different from the target inspection room. In the present embodiment, the connection destination confirmation section 13a compares the information stored in the memory and the transmission source device information, to determine the device correctness. If the ID information stored in the memory is different from the ID information from the video processor apparatus 20, the AI information confirmation section 13b determines that the transmission source device is not correct, which prevents an incorrect operation assistance to be provided to the user.

In addition, as described above, the transmission source device information received by the connection destination confirmation section 13a not only include the ID information of the video processor apparatus 20 but also include the action information and the operation information of the video processor apparatus 20 that include the information on the endoscope to be connected, the information related to the inspection, or the like, and further include the information related to the video format, etc. The connection destination confirmation section 13a may determine the device correctness based on the action information, the operation information, and the information related to the video format, etc. Furthermore, the connection destination confirmation section 13a may determine the device correctness using the information from the AI information confirmation section 13b and the result of the AI determination.

For example, the connection destination confirmation section 13a may determine that the device is not correct, when the AI information confirmation section 13b determines that the same image is continuously inputted in a case where the endoscopic image, as the input information, should change according to the change of the operation of the video processor apparatus 20. The connection destination confirmation section 13a may determine that the device is correct, when the AI information confirmation section 13b determines that the image has changed. In addition, the connection destination confirmation section 13a may determine that the device is correct, when the information of the video format matches the information of the video format determined by the AI information confirmation section 13b, and may determine that the device is not correct, when the information of the video format does not match the information of the video format determined by the AI information confirmation section 13b. Thus, the connection destination confirmation section 13a can determine the device correctness based on the transmission source device information from the video processor apparatus 20 and the image recognition result by the AI information confirmation section 13b.

Furthermore, in the case where the connection destination confirmation section 13a receives, for example, the operation information indicating that an endoscopy has been started, when the recognition result that the input image has changed to the image obtained by picking up an image inside a body, that is, the inspection has been started is obtained based on the AI determination by the AI determination section 14, the connection destination confirmation section 13a may determine that the device is correct. In addition, the connection destination confirmation section 13a may determine the device correctness depending on, for example, whether the operation information related to the observation mode such as the NBI or the RDI, matches the observation mode in the AI determination result.

Thus, the video processor apparatus 20, which is a medical image generation device, can set the observation mode for the endoscope. A determination on whether the endoscopic image obtained under the observation mode set by the video processor apparatus 20 has been supplied, as the input information, to the inspection assistance apparatus 10 is made based on the AI determination result. In other words, the device correctness is determined by determining whether the medical image generation device which is the connection destination of the first communication route and the medical image generation device which is the connection destination of the second communication route are identical to each other.

Note that when determining that the device is not correct based on the device correctness determination, the connection destination confirmation section 13a may output a signal for stopping the action of the AI determination section 14 to the AI determination section 14.

### Various Determinations

The AI determination section 14 may be configured to be able to perform various AI determinations. For example, the AI determination section 14 may be configured to be able to output the operation guide information of the endoscope as the inspection assistance information. Further, the AI determination section 14 may be configured to be able to output the control information of the automatic insertion endoscope. Furthermore, the AI determination section 14 may be configured to be able to output the identification information of the lesion and the site of the organ.

When the input information confirmation section 13 obtains the device correctness determination indicating that the transmission source device is not correct or the input eligibility determination indicating that the input information is not suitable for the AI determination, the input information confirmation section 13 may control the AI determination section 14 not to output the inspection assistance information. In addition, in a case where the endoscope connected to the video processor apparatus 20 is an automatic control endoscope which is configured to allow automatic insertion, when the input information confirmation section 13 obtains the device correctness determination indicating that the transmission source device is not correct or the input eligibility determination indicating that the input information is not suitable for the AI determination, the input information confirmation section 13 may be configured to cause an emergency stop apparatus, not shown, to operate for stopping the automatic insertion of the automatic control endoscope.

Thus, the input information confirmation section 13 outputs the inspection assistance information, when determining that the transmission source device which is the connection destination of the first communication route and the transmission source device which is the connection destination of the second communication route are identical to each other. In addition, the input information confirmation section 13 outputs alert information related to the connection destination of the first communication route and the connection destination of the second communication route, when determining that the transmission source device which is the connection destination of the first communication route and the transmission source device which is the connection destination of the second communication route are different from each other.

### Working

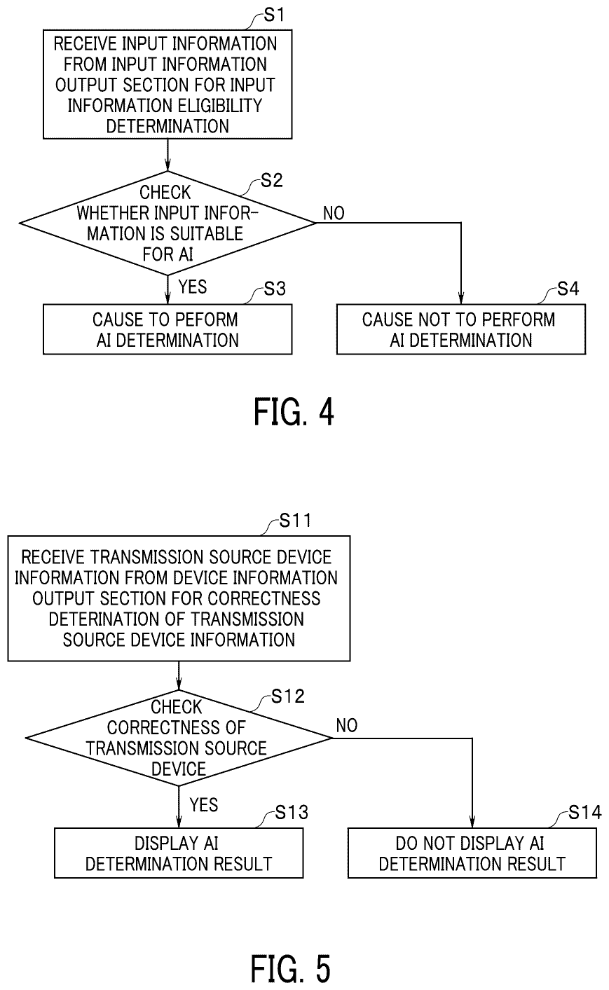

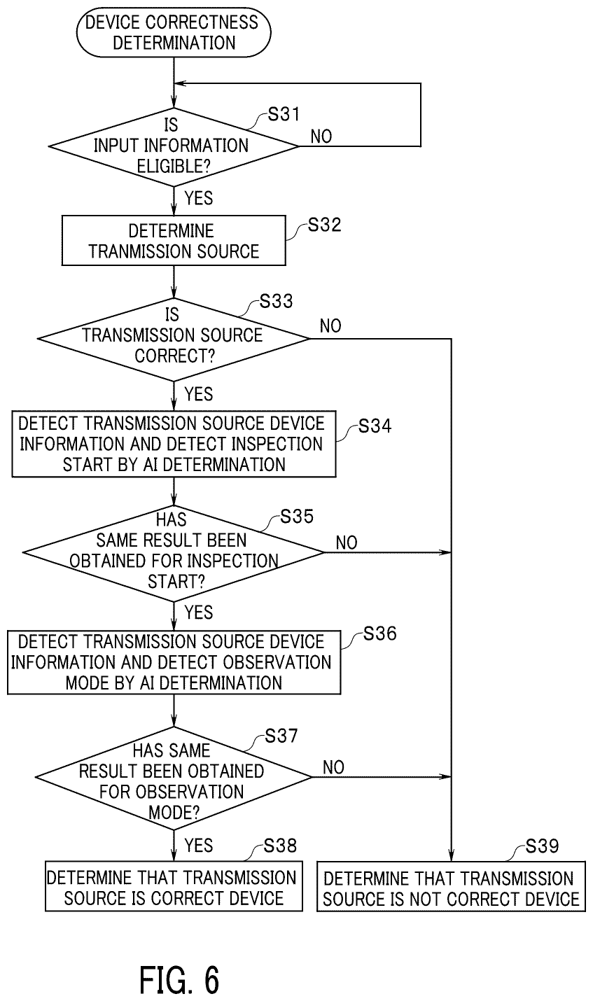

Next, working of the embodiment thus configured will be described with reference to FIGS. 4 to 6. FIG. 4 is a flowchart showing an AI determination control by the AI information confirmation section 13b. FIG. 5 and FIG. 6 are each a flowchart showing the device correctness determination by the connection destination confirmation section 13a.

The endoscope is connected to the video processor apparatus 20, and the endoscope is driven by the video processor apparatus 20, to obtain an endoscopic image. For example, in order to perform operation assistance for an endoscopy, the input information output section 22 of the video processor apparatus 20 outputs the endoscopic image obtained with the endoscope to the inspection assistance apparatus 10 via the second communication route. Furthermore, the device information output section 21 of the video processor apparatus 20 outputs the transmission source device information including the action information and the operation information of the video processor apparatus 20, the information of the video format, etc., of the endoscopic image, to the inspection assistance apparatus 10 via the first communication route.

In S1 in FIG. 4, the AI information confirmation section 13b receives the input information, from the input information output section 22 of the video processor apparatus 20, for the input eligibility determination of the input information. The AI information confirmation section 13b performs the image analysis of the medical image which is the received input information and the analysis of the metadata of the input information, to determine whether the input information is the image suitable for the AI determination (S2). If the AI information confirmation section 13b determines that the input information is the image suitable for the AI determination (YES determination in S2), the AI information confirmation section 13b outputs, to the AI determination section 14, the input eligibility determination result for causing the AI determination section 14 to perform the AI determination (S3). Furthermore, if the AI information confirmation section 13b determines that the input information is not the image suitable for the AI determination (NO determination in S2), the AI information confirmation section 13b outputs, to the AI determination section 14, the input eligibility determination result for causing the AI determination section 14 not to perform the AI determination (S4).

In this case, the AI determination section 14 does not perform the AI determination. In addition, the input information confirmation section 13 displays the warning messages such as “This is not the target endoscope”, and “The setting is different” on the display screen of the display section 30.

The AI information confirmation section 13b provides the input information to the AI determination section 14, if determining that the input information is suitable for the AI determination. The AI determination section 14 performs the recognition processing on the input information, to obtain the AI determination result. The AI determination section 14 outputs the AI determination result to the input information confirmation section 13.

The connection destination confirmation section 13a of the input information confirmation section 13 receives, in S11 in FIG. 5, the transmission source device information from the device information output section 21 of the video processor apparatus 20 for the device correctness determination of the transmission source device. The connection destination confirmation section 13a compares the received transmission source device information with the information stored in the memory, to determine whether the transmission source device that has transmitted the transmission source device information is the target device (S12). If the connection destination confirmation section 13a determines that the transmission source device is correct (YES determination in S12), the connection destination confirmation section 13a displays the AI determination result from the AI determination section 14 on the display section 30 (S13). On the other hand, if the connection destination confirmation section 13a determines that the transmission source device is not correct (NO determination in S12), the connection destination confirmation section 13a does not display the AI determination result from the AI determination section 14 on the display section 30 (S14).

In the example shown in FIG. 5, the connection destination confirmation section 13a determines the correctness of the transmission source device based on, for example, the ID information of the transmission source device, and the like, to decide whether to display or not to display the AI determination result. However, alternatively, the connection destination confirmation section 13a may determine the correctness of the transmission source device by using the outputs of the AI information confirmation section 13b and the AI determination section 14, to decide whether to display or not to display the AI determination result.

FIG. 6 shows the example in this case. The AI information confirmation section 13b of the input information confirmation section 13 determines, in S31 in FIG. 6, whether the input information is suitable for the AI determination. If the input information is not suitable for the AI determination, the AI determination is not performed, as described above. If the input information is suitable for the AI determination, the AI determination is performed and the AI determination result is provided to the input information confirmation section 13.

The input information confirmation section 13 determines, in S32, the correctness of the transmission source device based on the ID information and the like (transmission source determination). If determining that the transmission source device is correct (YES determination in S33), the input information confirmation section 13 determines in the next S34 whether the inspection has been started. In other words, if the input information confirmation section 13 detects that the inspection has been started based on the transmission source device information, the input information confirmation section 13 obtains, from the AI determination section 14, the AI determination result indicating whether the inspection has been started. If both the transmission source device information and the AI determination result indicate that the inspection has been started (YES determination in S35), the input information confirmation section 13 determines the observation mode in the next S36.

When the input information confirmation section 13 detects the observation mode based on the transmission source device information, the input information confirmation section 13 obtains, from the AI determination section 14, the AI determination result for the observation mode. If both the transmission source device information and the AI determination result indicate the same observation mode (YES determination in S37), the input information confirmation section 13 determines that the transmission source is the correct device in the next S38. In this case, the input information confirmation section 13 displays the AI determination result from the AI determination section 14 on the display section 30.

On the other hand, if the input information confirmation section 13 obtains NO determination in S33, S35, or S37, the input information confirmation section 13 determines that the transmission source is not the correct device in S39. In this case, the input information confirmation section 13 does not display the AI determination result from the AI determination section 14 on the display section 30. Note that, in this case, the input information confirmation section 13 displays the warning messages such as “This is not the target endoscope” and “The setting is different” on the display screen of the display section 30.

Thus, in the present embodiment, determination is made not only on the correctness of the transmission source device but also on the eligibility of the input information, which enables the reliable determination that the input information suitable for the AI determination is inputted from the target device. Such a configuration can prevent an unsuitable AI determination result from being displayed in cases that may occur due to an erroneous connection, etc., where the image which is not suitable for the AI processing is inputted or the AI determination is performed on the input information from the device which is not the target device.

### Modified Example

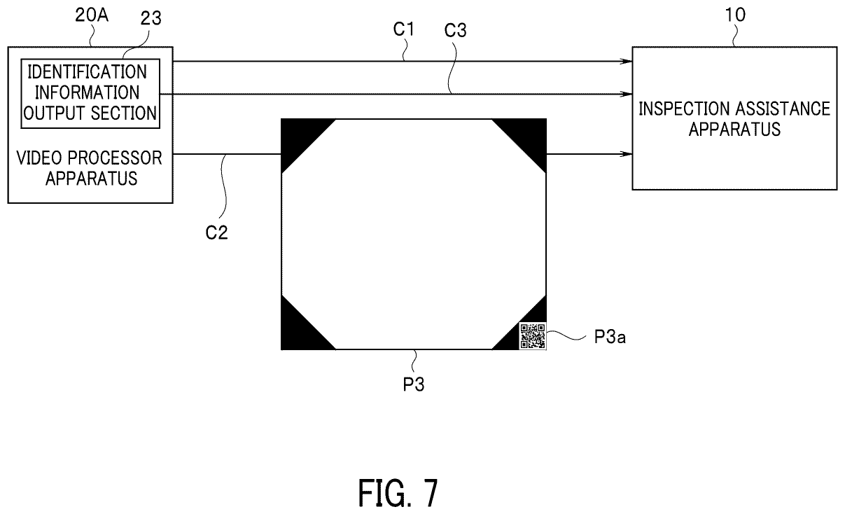

FIG. 7 is a block diagram showing a modified example. In FIG. 1, description has been made on the example in which the video processor apparatus 20 transmits the transmission source device information and the input information to the inspection assistance apparatus 10. In the modified example in FIG. 7, in the case where the identification information is transmitted in the manner superimposed on the input information, information similar to this identification information is transmitted to the inspection assistance apparatus 10 via a third communication cable C3 configuring a third communication route.

A video processor apparatus 20A is configured by additionally providing an identification information output section 23 to the video processor apparatus 20. When the identification information output section 23 transmits the input information added with the identification information from the device information output section 21, the identification information output section 23 outputs identification information having the same content as the identification information added to the input information to the inspection assistance apparatus 10 via the third communication cable C3. The device information acquisition section 11 of the inspection assistance apparatus 10 receives the identification information outputted from the identification information output section 23, to provide the received identification information to the input information confirmation section 13.

Note that the example has been shown above, in which the identification information output section 23 outputs the identification information to the inspection assistance apparatus 10 via the third communication cable C3. However, the third communication cable C3 may be omitted, and the identification information may be supplied to the inspection assistance apparatus 10 via the first communication cable C1.

The device information acquisition section 11 of the inspection assistance apparatus 10 provides the received identification information to the AI information confirmation section 13b. The AI information confirmation section 13b compares the identification information added to the input information with the identification information received via the third communication cable C3. When determining that these pieces of identification information have the same contents, the AI information confirmation section 13b may determine that the transmission source based on the input information matches the transmission source based on the transmission source device information, and in some cases, may further determine that the input information is suitable for the AI determination.

Other configurations and working are the same as those in the embodiment shown in FIG. 1.

According to the modified example thus configured, the pieces of identification information having the same contents are transmitted via the first communication route and the second (or third) communication route, which enables the correctness of the transmission source of the input information to be determined relatively easily.

The present disclosure is not limited to the above-described embodiments as they are, and it goes without saying to embody the disclosure by modifying the constituent elements in a range without departing from the gist of the disclosure at the practical stage. In addition, various disclosures can be achieved by appropriately combining the plurality of constituent elements disclosed in each of the above-described embodiments. Some of the constituent elements may be deleted from all the constituent elements shown in the embodiment, for example. Furthermore, constituent elements over different embodiments may be appropriately combined.

What is claimed is:

1. An inspection assistance system comprising,
one or more processors including hardware,
the one or more processors being configured to:
acquire a video signal including information of a medical image;
perform recognition processing based on the video signal, to enable outputting of inspection assistance information for assisting an inspection;
acquire identification information included in the video signal, the identification information being different from the information of the medical image; and
determine whether the video signal is a video signal for the recognition processing, based on the identification information.

2. The inspection assistance system according to claim 1, wherein
the one or more processors are configured to:
acquire transmission source device information related to a transmission source device via a first communication route connected to the transmission source device, which is a transmission source of the medical image obtained by photographing an image of a subject, to determine whether the transmission source device as a connection destination is suitable, based on the transmission source device information; and
acquire the video signal via a second communication route connected to the transmission source device, and determine, based on the acquired video signal, whether the video signal is the video signal for the recognition processing.

3. The inspection assistance system according to claim 1, wherein
the one or more processors are configured to determine whether the video signal is the video signal for the recognition processing, based on a position of the medical image on an image obtained by converting the video signal.

4. The inspection assistance system according to claim 1, wherein
the one or more processors are configured to determine whether the video signal is the video signal for the recognition processing based on an aspect ratio of the medical image.

5. The inspection assistance system according to claim 1, wherein
the one or more processors are configured to determine whether the video signal is the video signal for the recognition processing based on an image quality of the medical image.

6. The inspection assistance system according to claim 1, wherein
the one or more processors are configured to determine whether the video signal is the video signal for the recognition processing based on whether an image different from the medical image is superimposed on the medical image when the video signal is converted into an image.

7. The inspection assistance system according to claim 1, wherein
the one or more processors are configured to perform the recognition processing when the video signal is suitable.

8. The inspection assistance system according to claim 7, wherein
the identification information is a QR code.

9. The inspection assistance system according to claim 7, wherein
the one or more processors are configured to:
acquire information related to a content of the identification information via a first communication route connected to a transmission source device which is a transmission source of the medical image;
acquire the identification information via a second communication route connected to the transmission source device; and
determine whether a transmission source device which is a connection destination of the first communication route and a transmission source device which is a connection destination of the second communication route are identical to each other, based on the information related to the content of the identification information acquired via the first communication route and the identification information acquired via the second communication route.

10. The inspection assistance system according to claim 2, wherein
the transmission source device is a medical image generation device configured to set an observation mode and generate the medical image by observing the subject under the set observation mode, and
the one or more processors are configured to:
acquire information related to a setting of the observation mode, via the first communication route;
acquire the video signal via the second communication route; and
determine whether the medical image generation device which is a connection destination of the first communication route and the medical image generation device which is a connection destination of the second communication route are identical to each other based on whether the video signal corresponds to the setting of the observation mode.

11. The inspection assistance system according to claim 1, wherein
the one or more processors are configured to output an alert when determining that the video signal is not the video signal for the recognition processing.

12. The inspection assistance system according to claim 1, wherein
the one or more processors are configured:
not to output the inspection assistance information when determining that the video signal is not the video signal for the recognition processing; and
output the inspection assistance information when determining that the video signal is the video signal for the recognition processing.

13. The inspection assistance system according to claim 9, wherein
the one or more processors are configured to:
output the inspection assistance information when determining that the transmission source device which is the connection destination of the first communication route and the transmission source device which is the connection destination of the second communication route are identical to each other; and
output alert information related to the connection destination of the first communication route and the connection destination of the second communication route, when determining that the transmission source device which is the connection destination of the first communication route and the transmission source device which is the connection destination of the second communication route are different from each other.

14. The inspection assistance system according to claim 10, wherein
the one or more processors are configured to:
output the inspection assistance information when determining that the transmission source device which is the connection destination of the first communication route and the transmission source device which is the connection destination of the second communication route are identical to each other; and
output alert information related to the connection destination of the first communication route and the connection destination of the second communication route, when determining that the transmission source device which is the connection destination of the first communication route and the transmission source device which is the connection destination of the second communication route are different from each other.

15. The inspection assistance system according to claim 1, wherein
the one or more processors are configured to:
perform the recognition processing by inputting the video signal into a recognition processing apparatus which has performed learning with a medical image for learning; and
determine that the video signal is not the video signal for the recognition processing in at least one of: a case where at least one of an aspect ratio or an image quality of the medical image is different from an aspect ratio or an image quality of the medical image for learning; or a case where another image is superimposed on the medical image.

16. The inspection assistance system according to claim 1, wherein
the one or more processors are configured to output operation guide information of an endoscope as the inspection assistance information.

17. The inspection assistance system according to claim 1, wherein
the one or more processors are configured to output control information of an automatic control endoscope as the inspection assistance information.

18. An inspection assistance method comprising:
acquiring a video signal including information of a medical image;
acquiring identification information included in the video signal, the identification information being different from the information of the medical image;
determining whether the video signal is a video signal for recognition processing based on the identification information; and
performing the recognition processing based on the video signal, to output inspection assistance information for assisting an inspection using the medical image.

19. A control method for a medical device, the control method comprising:
acquiring transmission source device information related to a transmission source device which is a transmission source of a medical image:
determining whether the transmission source device as a connection destination is suitable, based on the transmission source device information;
acquiring a video signal including information of the medical image;
performing recognition processing based on the video signal, to enable outputting of inspection assistance information for assisting an inspection; and
determining, based on the video signal, whether the video signal is a video signal for the recognition processing.

20. A computer-readable recording medium on which a program is recorded,
the program causing a computer to execute:
acquiring a transmission source device information related to a transmission source device which is a transmission source of a medical image;
determining whether the transmission source device as a connection destination is suitable, based on the transmission source device information;
acquiring a video signal including information of the medical image;
performing recognition processing based on the video signal, to enable outputting of inspection assistance information for assisting an inspection; and
determining, based on the video signal, whether the video signal is a video signal for the recognition processing.
